(19) United States
(12) Patent Application Publication
(10) Pub. No.: US 2025/0205470 A1
(43) Pub. Date: Jun. 26, 2025

(54) SENSORS FOR PERCUTANEOUS PNEUMATIC CARDIAC ASSISTANCE SYSTEMS

(71) Applicant: PercAssist, Inc., Santa Clara, CA (US)

(72) Inventors: Hilman Hayming CHUNG, Mountain View, CA (US); Jeffrey STEWART, Bainbridge island, WA (US); Thi-Vu HUYNH, Saratoga, CA (US); Liyang XIE, Austin, TX (US)

(73) Assignee: PercAssist, Inc., Santa Clara, CA (US)

(21) Appl. No.: 19/075,525

(22) Filed: Mar. 10, 2025

Related U.S. Application Data
(63) Continuation of application No. PCT/US23/74243, filed on Sep. 14, 2023.
(60) Provisional application No. 63/407,100, filed on Sep. 15, 2022.

(51) Int. Cl. A61M 60/165; A61M 60/289; A61M 60/497; A61M 60/515; A61M 60/892; A61M 60/90

(52) U.S. Cl. CPC A61M 60/165; A61M 60/289; A61M 60/497; A61M 60/515; A61M 60/892; A61M 60/90; A61M 2205/15; A61M 2205/3303; A61M 2205/3327; A61M 2205/3331; A61M 2205/50; A61M 2210/125; A61M 2230/005; A61M 2230/04

(57) ABSTRACT

A cardiac assist system includes a pneumatic effector and an external drive unit. The pneumatic effector is implanted proximate a patient's heart to enhance left ventricular contraction. The external drive unit includes a gas pump connectable to the pneumatic effector and is configured to actuate the pneumatic effector assembly in response to the patient's sensed heart rhythm. The system may include an isolation valve located between the gas pump assembly and an inlet to the pneumatic effector and a pressure sensor located between the isolation valve and the inlet to the pneumatic effector, where the control circuitry is configured to receive changes in pressure sensed by the pressure sensor the when the isolation valve is closed to isolate the pneumatic effector. The system may also include a liquid accumulator and a liquid sensor adjacent the liquid accumulator sensor to detect liquid in the pneumatic effector.

CROSS REFERENCE

This application is a continuation of PCT/US23/74243, filed Sep. 14, 2023, which claims the benefit of U.S. Provisional Application No. 63/407,100, filed on Sep. 15, 2022, which is incorporated herein by reference in its entirety.

### BACKGROUND

1. Field of the Invention. The present invention relates generally to medical devices, systems, and methods. More particularly, the present invention relates to systems for providing cardiac assist to patients suffering from late-stage heart failure.

2. Description of the Background Art. WO2020/176670, commonly assigned with the present application, describes a “ventricular assist” balloon cannula configured to be inserted inside a patient's pericardial sac and positioned anterior to the patient's left ventricle. Inflation of the balloon during cardiac systole and deflation of the balloon during cardiac diastole is performed to increase cardiac output in patients with congestive heart failure and other conditions.

The ventricular assist balloon cannula is typically inserted through the pericardium at the inferior aspect of the heart near the apex, typically via a percutaneous subxiphoid incision or needle puncture. A distal end of the ventricular assist balloon cannula is advanced to the left lateral aspect of the heart immediately inferior to the left atrial appendage to position the balloon anterior to the left ventricle.

A fluid tight reservoir is then attached to the proximal end of the ventricular assist balloon cannula, and the reservoir implanted subcutaneously in the subxiphoid region. The balloon is inflated with air or other gas via an external pump using a large bore needle penetrated through the patient's skin and into the subcutaneous reservoir. A gaseous inflation media is used as it allows rapid inflation and deflation which is necessary to maintain heart rhythm. Any leak from or into the air pumping circuit would be problematic, including any pressure loss or intrusion of a liquid.

While pressure loss can be monitored directly, small leaks can be difficult to detect as the system pressure measurements are often noisy. Liquid intrusion can be difficult to detect as liquids will tend to pool in locations where their presence may be difficult to sense.

It would therefore be desirable to provide improved apparatus, system, and methods for detecting or sensing both pressure loss and fluid intrusion in the air pumping systems of ventricular assist balloon cannula systems of the type described in WO2020/176670. It would be further desirable if such apparatus, system, and method would find use in other percutaneous systems of the having pumping systems, such as left atrial assist devices (LVAD's), intra-aortic balloon pumps (IABP's), and the like. At least some of these objectives will be met by the inventions described below.

### SUMMARY

In accordance with the principles of the present invention, a cardiac assist system includes a pneumatic effector assembly and an external drive unit. A balloon or other pneumatic effector of the pneumatic effector system is implanted proximate a patient's heart to enhance heart contraction and pumping, such as left ventricular contraction, and the external drive unit includes a gas pump connectable to the pneumatic effector assembly and configured to actuate the pneumatic effector in response to the patient's sensed heart rhythm. In some embodiments, the cardiac assist systems may include an isolation valve located between the gas pump assembly and an inlet to the pneumatic effector and a pressure sensor located between the isolation valve and the inlet to the pneumatic effector, where the control circuitry is configured to receive changes in pressure sensed by the pressure sensor the when the isolation valve is closed to isolate the pneumatic effector. In some embodiments, the cardiac assist systems may include a liquid accumulator and a liquid sensor adjacent the liquid accumulator sensor to detect liquid in the pneumatic effector. In some embodiments, the cardiac assist systems may include both the pressure sensing and liquid accumulation detection features.

In a first aspect, the present invention provides a cardiac assist system comprising a pneumatic effector assembly, external drive unit, and a sensor assembly or sub-system for monitoring system performance during use. The pneumatic effector assembly is typically configured to be implanted proximate a patient's heart to enhance left ventricular contraction. The external drive unit typically includes (a) a gas pump assembly connectable to the pneumatic effector assembly and (b) control circuitry configured to operate the gas pump assembly to actuate the pneumatic effector assembly in response to the patient's sensed heart rhythm.

In a first embodiment, the sensor assembly or sub-system is configured to measure pressure in the pneumatic effector, typically intended particularly for leak detection. For example, a pressure sensing sub-system may comprise an isolation valve located between the gas pump assembly and an inlet to the pneumatic effector. A pressure transducer or other sensor is located between the isolation valve and the inlet to the pneumatic effector, and the control circuitry is configured to receive changes in pressure sensed by the pressure sensor when the isolation valve is closed to isolate the pneumatic effector. Closing the isolation valve thus isolates the pneumatic effector assembly so that any loss of pressure indicates a breach or other failure in the pneumatic effector assembly.

In specific instances, the isolation valve may be located within an external drive unit housing together with the gas pump assembly and the control circuitry. In other instances, the isolation valve may be located between an external drive unit housing and the pneumatic effector assembly.

In a second embodiment, the sensor assembly or sub-system comprises (1) a liquid accumulator located between an outlet of the gas pump assembly and an inlet of the pneumatic effector assembly and (2) a liquid sensor adjacent to the liquid accumulator sensor. The liquid accumulator is configured to collect liquids that might enter the pneumatic effector assembly or otherwise have intruded into the air pumping circuit. Such liquid intrusion could result from a breach in the pneumatic effector assembly in which case there would likely be a pressure loss or could be entering from other sources with a noticeable pressure loss. Thus, while the pressure and liquid intrusion sensor sub-systems are independent, they are also complementary and both indicative of a system failure.

In specific examples, the pneumatic effector assembly of the pressure sensing embodiments may comprise a catheter body having a proximal end, a distal end, and at least one gas exchange lumen therebetween. The pneumatic effector is typically located at the distal end of the catheter body configured (1) to be implanted proximate to a patient's heart to enhance left ventricular contraction and (2) to receive and exhaust an inflation gas through the gas exchange lumen. A hub may be located at the proximal end of the catheter body and may be configured to be detachably connected to the gas pump assembly of the external drive unit. The pressure sensor may be located in or on the hub and may be configured to sense pressure in the at least one gas exchange lumen. The hub may be attached directly to the proximal end of the catheter body (when the catheter body is intended for percutaneous introduction) but will more often comprise a cannula to percutaneously access an implantable port attached subcutaneously (directly) to the proximal end of the catheter body, where said implantable port is configured to percutaneously receive the cannula.

In specific examples, the control circuitry may be configured to calculate a pressure change gradient of any change in pressure received from the pressure sensor during a pre-determined pressure measurement period. For example, a calculated pressure change gradient (rate of change measured over a small time interval, typically from 50 msec to 250 msec, usually from 100 msec to 150 msec) larger than a predetermined threshold will typically indicate that there is a pressure leak in the pneumatic effector assembly. The pressure-change gradient is considered to be a more sensitive indicator of pressure loss that the pressure loss value itself.

In specific examples, the pneumatic effector assembly of the liquid intrusion sensing embodiments may comprise a catheter body having a proximal end, a distal end, and at least one gas exchange lumen therebetween. A pneumatic effector at the distal end of the catheter body is configured (1) to be implanted proximate a patient's heart to enhance left ventricular contraction and (2) to receive and exhaust an inflation gas through the gas exchange lumen. A hub at the proximal end of the catheter body is configured to be detachably connected to the gas pump assembly of the external drive unit, where the liquid accumulator is located in or on the hub and is configured to accumulate liquid from the at least one gas exchange lumen and the liquid sensor is configured to detect liquid that has accumulated in the liquid accumulator. The hub may be attached directly to the proximal end of the catheter body (when the catheter body is intended for percutaneous introduction) but will more often comprise a cannula to percutaneously access an implantable port attached directly to the proximal end of the catheter body, where said implantable port is configured to percutaneously receive the cannula.

In specific examples, the liquid accumulator may comprise a flow path between gas pump assembly and the pneumatic effector, where flow path may have baffles and/or a narrowed cross-sectional area to collect liquid entrained in gas flowing through the flow path. In such instances, the liquid sensor is typically located in the hub adjacent to the baffles and/or narrowed cross-sectional area of the flow path.

Regardless of sensor type, the cardiac assist systems may further comprise a connecting tube having a pump end attachable to the gas pump assembly and a hub end attachable to the hub, where the connecting tube connects the gas pump assembly to the pneumatic effector assembly. The pneumatic effector assembly may be configured to be implanted beneath a patient's pericardial sac and over a myocardial surface overlying the patient's left ventricle. The pneumatic effector assembly may be configured to be implanted in a heart chamber. The cardiac assist system may further comprise at least one ECG electrode, where the at least one ECG electrode may be located on the pneumatic effector assembly and configured to provide the control circuitry with the patient's heart rhythm. Alternatively, the at least one ECG electrode may be configured to attach externally to the patient and is connected to said ECG circuitry by an external lead.

In a second aspect, the present invention provides a method for assisting cardiac function comprising detecting a patient's ECG to determine a cardiac rhythm and delivering a driving gas from an external gas pump assembly to a pneumatic effector assembly implanted proximate a patient's heart to enhance left ventricular contraction. Driving gas delivery is synchronized with the determined cardiac rhythm to cause the pneumatic effector assembly to compress the heart and/or expand within a left ventricle of the heart at a rate which matches the cardiac rhythm. The external pump assembly is isolated from the pneumatic effector assembly to prevent any exchange of the driving gas, and pressure in the pneumatic effector assembly is measured while said pneumatic effector assembly is isolated from the external pumping assembly. By detecting a change in the measured pressure, potential leaks in the pneumatic effector assembly may be detected.

In specific instances, isolating the external pump assembly from the pneumatic effector assembly may comprise closing an isolation valve disposed between the external gas pump assembly and the pneumatic effector assembly. Typically, the isolation valve is closed at the end of deflation during systole and opened at the beginning of inflation during diastole. Typically, pressure in the pneumatic effector assembly is measured by a pressure sensor located between the isolation valve and the inlet to the pneumatic effector.

In specific instances, the control circuitry receives a pressure reading or value from the pressure sensor and determines whether the changes in pressure exceed a threshold value indicating a gas leak in the pneumatic effector assembly. In preferred instances, the control circuitry calculates a gradient of the pressure change and a gradient value that exceeds a threshold value indicates a gas leak in the pneumatic effector assembly.

In specific instances, the pneumatic effector assembly is implanted beneath the patient's pericardial sac and over a myocardial surface.

In specific instances, the patient's heart rhythm by detecting ECG with one or more electrodes located on the pneumatic effector. Alternatively, the ECG is detected with an external electrode.

In a third aspect, the present invention provides method for assisting cardiac function comprising: detecting a patient's ECG to determine a cardiac rhythm and delivering a driving gas from an external gas pump assembly to a pneumatic effector assembly implanted proximate a patient's heart to enhance left ventricular contraction, where driving gas delivery is synchronized with the determined cardiac rhythm to cause the pneumatic effector assembly to compress the heart or expand within a left ventricle of the heart at a rate which matches the cardiac rhythm. Liquid in the air pumping circuit, if any, is accumulated in a location between an outlet of the gas pump assembly and an inlet of the pneumatic effector, typically using a liquid sensor adjacent the liquid accumulator sensor.

In some instances, the accumulator comprises a flow path between gas pump assembly and the pneumatic effector, where the flow path may have baffles and/or a narrowed cross-sectional area to collect liquid entrained in gas flowing through the flow path. In such instances, the liquid sensor is typically located in a hub adjacent to the baffles and/or narrowed cross-sectional area of the flow path.

In some instances, the pneumatic effector assembly is implanted beneath the patient's pericardial sac and over a myocardial surface. In some instances, the patient's cardiac rhythm is detected using ECG electrodes which may be located on the pneumatic effector or on an external electrode.

In a fourth aspect, the present invention provides an implantable cardiac assist catheter for use with an external drive unit. The implantable cardiac assist catheter comprises a catheter body having a proximal end, a distal end, and at least one gas exchange lumen therebetween. A pneumatic effector is disposed at the distal end of the catheter body and is configured (1) to be implanted proximate a patient's heart to enhance left ventricular contraction and (2) to receive and exhaust an inflation gas through the gas exchange lumen. A hub is located at the proximal end of the catheter body and is configured to be detachably connected to a gas pump assembly of the external drive unit. A pressure sensor is configured to be detachably connected control circuitry in the external drive unit to measure pressure in the pneumatic effector.

In specific instances, the hub is attached directly to the proximal end of the catheter body, where the hub may further comprise a cannula and the pneumatic effector assembly may include an implantable port attached directly to the proximal end of the catheter body, wherein said implantable port is configured to percutaneously receive the cannula. A connecting tube having a pump end attachable to the gas pump assembly and a hub end attachable to the hub is typically provided to connect the gas pump assembly to the pneumatic effector assembly. The pneumatic effector assembly is typically configured to be implanted beneath a patient's pericardial sac and over a myocardial surface overlying the patient's left ventricle. The pneumatic effector assembly is typically configured to be implanted in a heart chamber, and the catheter may further comprise at least one ECG electrode. For example, at least one ECG electrode may be located on the pneumatic effector assembly and configured to provide the control circuitry with the patient's heart rhythm.

In a fifth aspect, the present invention provides an implantable cardiac assist catheter for use with an external drive unit. The implantable cardiac assist catheter comprises a catheter body having a proximal end, a distal end, and at least one gas exchange lumen therebetween. A pneumatic effector at the distal end of the catheter body is configured (1) to be implanted proximate a patient's heart to enhance left ventricular contraction and (2) to receive and exhaust an inflation gas through the gas exchange lumen. A hub at the proximal end of the catheter body is configured to be detachably connected to a gas pump assembly of the external drive unit, and a liquid accumulator is located between an outlet of the gas pump assembly and an inlet of the pneumatic effector. A liquid sensor adjacent to the liquid accumulator sensor.

In some embodiments, the hub may be attached directly to the proximal end of the catheter body, allowing direct percutaneous implantation of the catheter. More often, however, the hub further comprises a cannula and the pneumatic effector assembly includes an implantable port attached directly to the proximal end of the catheter body so that implantable port can percutaneously receive the cannula.

In specific instances, the implantable cardiac assist catheter further comprises a connecting tube having a pump end attachable to the gas pump assembly and a hub end attachable to the hub, wherein the connecting tube connects the gas pump assembly to the pneumatic effector assembly.

The pneumatic effector assembly is typically configured to be implanted beneath a patient's pericardial sac and over a myocardial surface overlying the patient's left ventricle. Alternatively, the pneumatic effector assembly may be configured to be implanted in a heart chamber. The implantable cardiac assist catheter will typically further comprising at least one ECG electrode, e.g., being located on the pneumatic effector assembly and configured to provide the control circuitry with the patient's heart rhythm.

In a sixth aspect, the present invention provides an external drive unit (EDU) for use with an implantable cardiac assist catheter having a pneumatic effector assembly and a pressure sensor, said external drive unit comprising. The EDU comprises a gas pump assembly having a port connectable to the pneumatic effector assembly, control circuitry configured to operate the gas pump assembly to actuate the pneumatic effector assembly in response to a patient's sensed heart rhythm, and an isolation valve located between an outlet of the gas pump assembly and the port. The control circuitry is typically configured to receive changes in pressure sensed by the pressure sensor when the isolation valve is closed to isolate the pneumatic effector.

In a seventh aspect, the present invention provides an external drive unit (EDU) for use with an implantable cardiac assist catheter having a pneumatic effector assembly and a liquid accumulation sensor. The EDU comprises a gas pump assembly having a port connectable to the pneumatic effector assembly and control circuitry configured to operate the gas pump assembly to actuate the pneumatic effector assembly in response to a patient's sensed heart rhythm. The control circuitry is configured to receive an output of the liquid accumulation sensor to detect when liquid is accumulating in the implantable cardiac assist catheter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a prior art cardiac assist system as described in WO2020/176670 implanted in a patient.

FIG. 2 is a schematic illustration of a cardiac assist system constructed in accordance with the principles of the present invention.

FIG. 3 is a cross-sectional view of a hub of the cardiac assist system of the present invention including both pressure sensing and fluid accumulation sensing components.

FIG. 3A is detailed view of an alternative structure of the accumulation sensing component of the present invention.

FIG. 4 is an isometric view of the hub of FIG. 3.

FIG. 4A is an end view of the hub of FIG. 3.

FIG. 5 is an isometric view of an alternative embodiment of the hub having a cannula configured to access an implanted port.

FIG. 6 illustrates the relationship between ECG monitoring and pressure sensing in accordance with the principles of the present invention.

### DETAILED DESCRIPTION

For purposes of this patent application, the term “distal” refers to the end of the device that is farthest away from the operator, and closest to the heart. This is also the “upstream” direction of blood flow. The term “proximal” refers to the end of the device nearer to the operator, toward the direction of the access site where the device has been introduced into the body, and farthest away from the heart. This is also the “downstream” direction of blood flow.

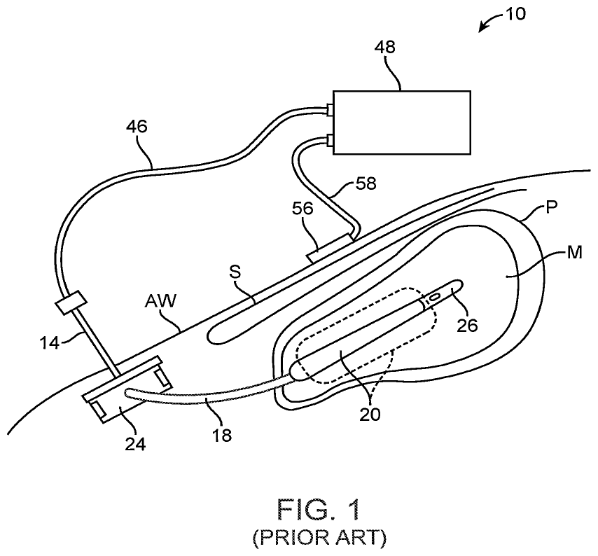

The apparatus, system, and methods of the present invention are intended as accessory features and improvement and improvements to the “ventricular assist” balloons described in WO2020/176670, commonly assigned with the present application, the full disclosure of which is incorporated herein by reference. As described in that PCT publication, for example, a cardiac assist system 10 as shown in FIG. 1 may be implanted in a patient as illustrated. A balloon or other pneumatic effector 20 is introduced into the pericardial sac between an inner surface of the pericardium P and an outer surface of the myocardium M. The pneumatic effector 20 will preferably be located generally over the left ventricle so that inflation or other actuation of the effector compresses the left ventricle, as shown in broken line. Implanted port 24 is connected to the balloon by catheter body 18 and accessed percutaneously by cannula 14. An external drive unit 48 delivers actuating gas through a connecting tube 46 and the cannula 14 to the port 24 in order to actuate the pneumatic effector, typically by inflating and deflating the pneumatic effector. ECG is measured by the ECG pad 56 which is connected to the external drive unit by cable 58. Optionally, other ECG signals may be measured by electrodes on the implantable port 24 or elsewhere in the system.

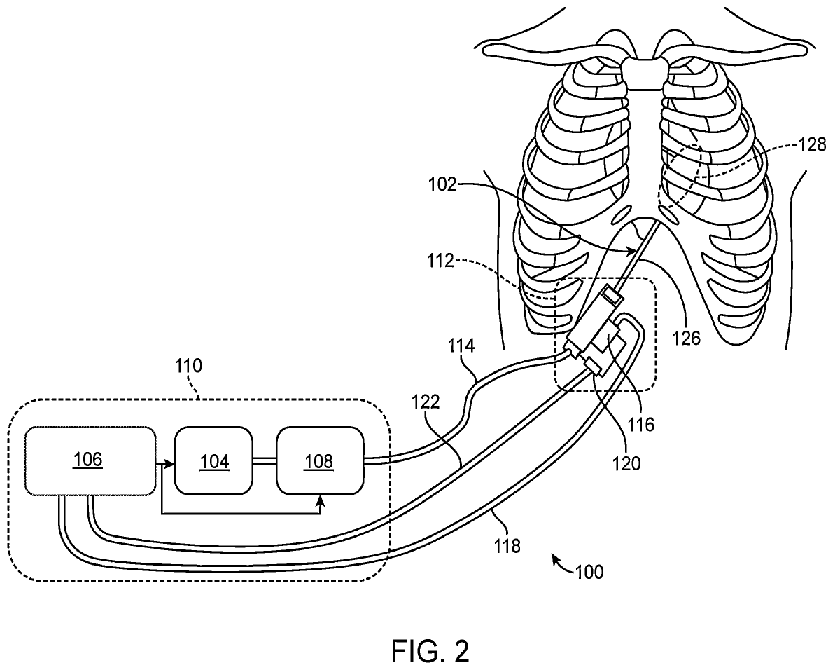

Referring now to FIG. 2, a cardiac assist system 100 constructed in accordance with the principles of the present invention will be described. the cardiac assist system comprises a pneumatic effector assembly 102, a gas pump assembly 104, and control circuitry 106. The gas pump assembly 104 and the control circuitry 106 will typically be enclosed in an external drive unit housing 110 which typically further includes an isolation valve 108. The isolation valve will typically be a solenoid valve but could be any electrically operated valve capable of on-off control of a gas stream.

The pneumatic effector assembly 102 is directly or indirectly connected to a hub 112. As shown in FIG. 2, the hub 112 is connected directly to a catheter shaft of the pneumatic effector assembly 102, and the shaft in turn is connected to a pneumatic effector 128, typically an inflatable balloon. Alternatively, for the hub 112 could be configured for indirect connection to a pneumatic effect or assembly of the type shown in the prior art cardiac assist system 10 urn FIG. 1. In such cases, the hub will include a cannula 162 configured to percutaneously penetrate the patient's skin, typically in the chest, to access an implanted port 24, as described above.

The external drive unit 110 is connected to the hub 112 by connecting lines and cables so that the external drive unit may be maintained at a distance from the patient during operation of the cardiac assist system 100. The isolation valve 108 is connected to an inlet port 150 by an inflation line 114, and the isolation valve 108, in turn, is connected to the gas pump assembly 104 so that inflation gas may be reciprocatably delivered to the pneumatic effector 128 in order to compress the heart, as described in WO2020/176670. The gas pump assembly 104 typically comprises a reciprocating pump, a peristaltic pump, or any other pump capable of delivering and removing gas from the pneumatic effector at a rate desired for cardiac assistance. The isolation valve 108 will be normally open while the gas pump assembly 104 is driving the pneumatic effector 128 but will be closed to isolate the entire pneumatic effector assembly 102 (including the pneumatic effector 128, the shaft 126, and the hub 112) when pressure is being measured, as described in more detail below.

A pressure sensor 116 in the hub 110 is connected to the control circuitry 106 in the external drive unit 110 by a pressure sensor cable 118. A liquid sensor 120 is connected to the control circuitry 106 by a liquid sensor cable 122. in this way, as described in more detail below, the external drive unit can check the pneumatic effector assembly 102 for breaches or failures that can result in either loss of gas pressure or liquid accumulation in the gas lines.

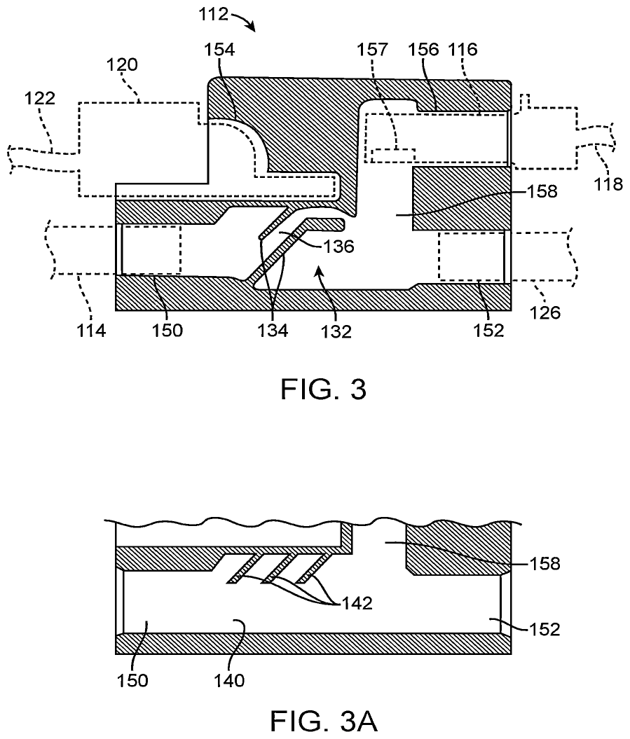

Referring now to FIG. 3, the internal construction of an exemplary hub 112 will be described. Typically, the hub 112 comprises a solid polymeric or other body having passages or cavities formed therein. A liquid accumulation structure 132 is formed in a gas flow path 133 between the gas inlet port 150 and the gas outlet port 152. As shown in FIG. 3, the liquid accumulator structure 132 may comprise baffles 134 forming a restricted flow path 136 therebetween. By restricting the flow of inflation into this flow path 136, any liquid entrained in the gas will collect and accumulate in the “upper” region of the passage as shown in FIG. 3. By further locating the liquid accumulation sensor 120 in a liquid sensor port 152 located adjacent to the restricted flow path 136, the ability to detect fluids is greatly enhanced.

The liquid accumulator structure may have a variety of different designs. For example, as shown in FIG. 3A, a liquid accumulator structure 140 may comprise a plurality of a baffled 142 arranged in a chevron pattern to collect liquid from the inflation gas as it flows past in the gas flow path 133.

Referring again to FIG. 3, the pressure sensor 116 may be located in a pressure sensor port 156 formed in the body of the hub 112. A pressure transducer element 157 on the pressure sensor 116 will be located adjacent to a pressure passage 158 which exposes the transducer to the pressure present in the gas flow line between gas inlet port 150 and gas outlet port 152.

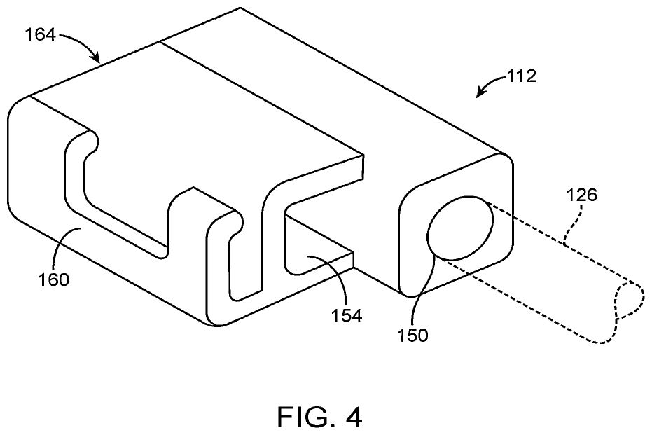

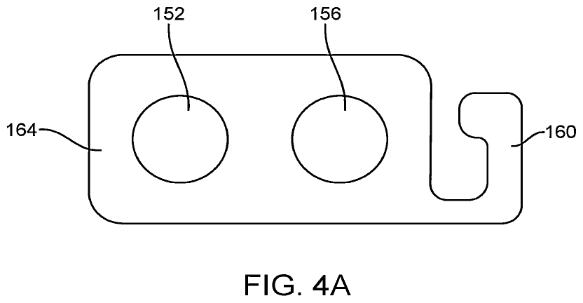

An external, isometric view of the hub 112 is shown in FIG. 4. The gas inlet port 150 is configured to removably or fixedly receive a proximal end of the catheter shaft 126, and a cable clip 160 is usually provided to hold the pressure sensor cable 118 in place as it is turned rearwardly to connect to the external drive unit 110, as seen in FIG. 2. The gas outlet port 152 and the pressure sensor port 156 are located on a back face 164 of the hub 112, as seen in FIG. 4A.

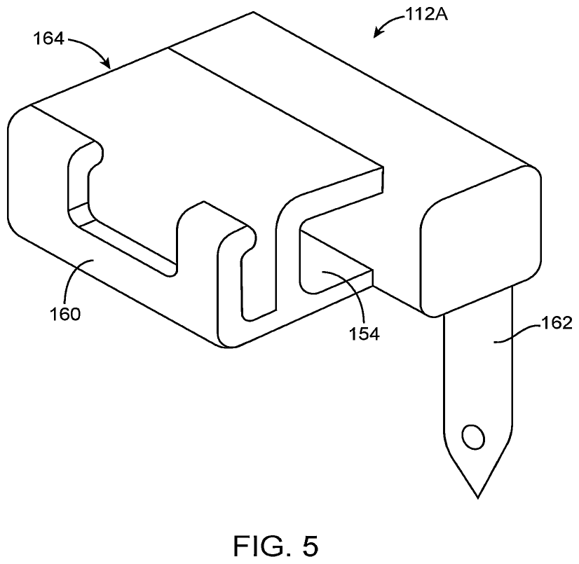

In a preferred example, as shown in FIG. 5, a hub 112a will include a cannula 162 configured for percutaneously connecting to an implanted port such as the implanted port 24 shown in FIG. 1. In such cases, the hub 112a will not need a gas inlet port 50, and the internal passages of the hub will be reconfigured so that gas will enter the gas flow path through the cannula 162.

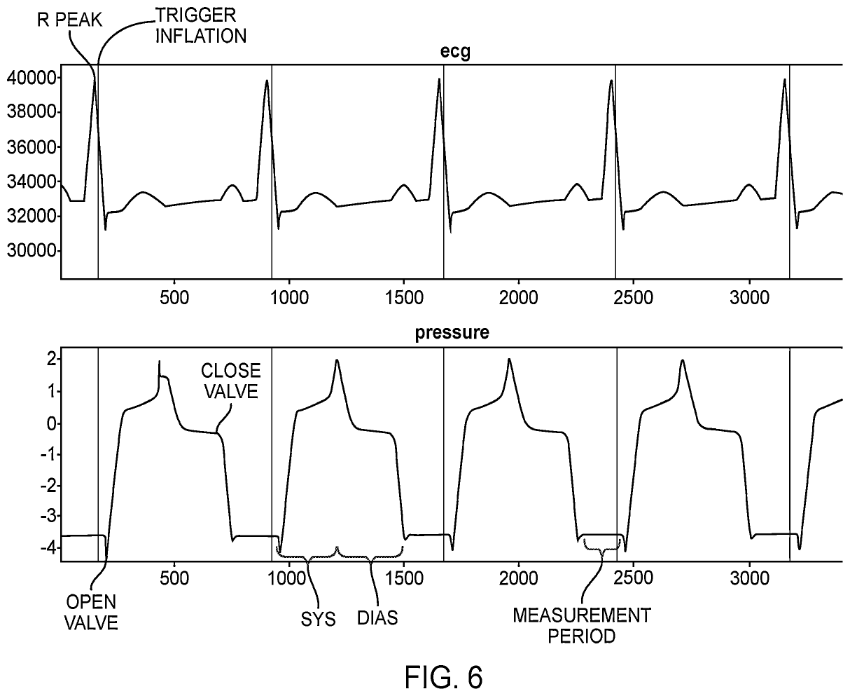

Referring now to FIG. 6, operation of the isolation valve 108 and the pressure sensor 116 for detecting pressure loss from the cardiac assist system 100 will be described. The patient's ECG and heart rate and will be monitored, typically using an external ECG sensor, for example as shown in FIG. 1. The isolation valve 108 will be open and the gas pump assembly 104 started when an “R Peak” is detected in the patient's ECG. The gas pump assembly 104 will inflate the pneumatic effector 128 during systole and then will be reversed to deflate the pneumatic effector during diastole. The isolation valve 108 will be closed at or near the end of diastole, and pressure will be measured from the end of diastole until the beginning of systole, shown as the “measurement period” in FIG. 6. As the pneumatic effector assembly 102 will be fully isolated by the isolation valve 108, any pressure loss detected by the pressure sensor 116 will result from a breach or failure in the pneumatic effector assembly 102.

In some instances, the control circuitry 106 may be programmed to detect the “gross” pressure loss over all or a portion of the measurement period between the end of diastole and the beginning of systole while the pneumatic effector assembly 102 is isolated. Often, however, it will be desirable to further calculate a gradient or derivative of the change in pressure at one or more times during the measurement period. While the overall pressure loss may be small, the gradient or rate of change in pressure may be relatively larger and easier to detect.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A cardiac assist system comprising:
a pneumatic effector assembly configured to be implanted proximate a patient's heart to enhance heart contraction;
an external drive unit including:
(a) a gas pump assembly connectable to the pneumatic effector assembly; and
(b) control circuitry configured to operate the gas pump assembly to actuate the pneumatic effector assembly in response to the patient's sensed heart rhythm;
an isolation valve located between the gas pump assembly and an inlet to the pneumatic effector; and
a pressure sensor located between the isolation valve and the inlet to the pneumatic effector;
wherein the control circuitry is configured to receive changes in pressure sensed by the pressure sensor when the isolation valve is closed to isolate the pneumatic effector.

2. The cardiac assist system of claim 1, wherein the isolation valve is located within an external drive unit housing.

3. The cardiac assist system of claim 1, wherein the isolation valve is located between an external drive unit housing and the pneumatic effector assembly.

4. The cardiac assist system of claim 1, wherein the pneumatic effector assembly comprises:
a catheter body having a proximal end, a distal end, and at least one gas exchange lumen therebetween;
a pneumatic effector at the distal end of the catheter body configured (1) to be implanted proximate a patient's heart to enhance left ventricular contraction and (2) to receive and exhaust an inflation gas through the gas exchange lumen; and
a hub at the proximal end of the catheter body configured to be detachably connected to a gas pump assembly of the external drive unit;
wherein the pressure sensor is located in or on the hub and configured to sense pressure in the at least one gas exchange lumen.

5. The cardiac assist system of claim 4, wherein the hub is attached directly to the proximal end of the catheter body.

6. The cardiac assist system of claim 4, wherein the hub further comprises a cannula and wherein the pneumatic effector assembly includes an implantable port attached directly to the proximal end of the catheter body, wherein said implantable port is configured to percutaneously receive the cannula.

7. The cardiac assist system of claim 1, wherein the control circuitry is configured to calculate a pressure change gradient of any change in pressure received from the pressure sensor.

8. The cardiac assist system of claim 7, wherein a calculated pressure change gradient larger than a predetermined threshold indicates that there is a pressure leak in the pneumatic effector assembly.

9. The cardiac assist system of claim 4, further comprising a connecting tube having a pump end attachable to the gas pump assembly and a hub end attachable to the hub, wherein the connecting tube connects the gas pump assembly to the pneumatic effector assembly.

10. The cardiac assist system of claim 1, wherein the pneumatic effector assembly is configured to be implanted beneath a patient's pericardial sac and over a myocardial surface overlying the patient's left ventricle.

11. The cardiac assist system of claim 1, wherein the pneumatic effector assembly is configured to be implanted in a heart chamber.

12. The cardiac assist system of claim 1, further comprising at least one ECG electrode.

13. The cardiac assist system of claim 12, wherein the at least one ECG electrode is located on the pneumatic effector assembly and configured to provide the control circuitry with the patient's heart rhythm.

14. The cardiac assist system of claim 12, wherein the at least one ECG electrode is configured to attach externally to the patient and is connected to said ECG circuitry by an external lead.

15. (canceled)

16. (canceled)

17. The cardiac assist system of claim 1, wherein the isolation valve is closed at the end of deflation during systole and opened at the beginning of inflation during diastole.

18-24. (canceled)

25. The cardiac assist system of claim 1, further comprising:
a liquid accumulator in-line between an outlet of the gas pump assembly and an inlet of the pneumatic effector; and
a liquid sensor adjacent the liquid accumulator sensor.

26. (canceled)

27. The cardiac assist system of claim 25, wherein the liquid accumulator comprises a flow path between gas pump assembly and the pneumatic effector, said flow path having baffles and/or a narrowed cross-sectional area to collect liquid entrained in gas flowing through the flow path.

28. The cardiac assist system of claim 25, wherein the liquid sensor is located in a hub adjacent to the baffles and/or narrowed cross-sectional area of the flow path.

29-48. (canceled)

49. An external drive unit for use with an implantable cardiac assist catheter having a pneumatic effector assembly and a pressure sensor, said external drive unit comprising:
a gas pump assembly having a port connectable to the pneumatic effector assembly;
control circuitry configured to operate the gas pump assembly to actuate the pneumatic effector assembly in response to a patient's sensed heart rhythm;
an isolation valve located between an outlet of the gas pump assembly and the port; and
wherein the control circuitry is configured to receive changes in pressure sensed by the pressure sensor the when the isolation valve is closed to isolate the pneumatic effector.

50. The external drive unit of claim 49,
wherein the control circuitry is configured to receive an output of a liquid accumulation sensor in the implantable cardiac assist catheter to detect when liquid is accumulating in the implantable cardiac assist catheter.
